(19) United States
(12) Patent Application Publication
(10) Pub. No.: US 2025/0057637 A1
(43) Pub. Date: Feb. 20, 2025

(54) 3D SCANNING METHOD AND APPARATUS FOR DENTAL APPLICATIONS

(71) Applicant: Rami Salloum, Bethlehem, PA (US)

(72) Inventor: Rami Salloum, Bethlehem, PA (US)

(21) Appl. No.: 18/805,896

(22) Filed: Aug. 15, 2024

Related U.S. Application Data
(60) Provisional application No. 63/520,182, filed on Aug. 17, 2023.

(51) Int. Cl. A61C 9/00; A61C 13/00

(52) U.S. Cl. CPC A61C 9/0046; A61C 13/0004; A61C 13/0019

(57) ABSTRACT

A method and device for aligning face scans with intraoral scans in dentistry applications. Digital intraoral scans capture the upper and lower arches and a bite registration. An Anterior Extension Device is affixed in the patient's mouth. A bio-copy of the upper arch scan is trimmed to remove the areas covered by the Anterior Extension Device. A rigid link extends between the Anterior Extension Device and base of the patient's nose. The bio-copy is rescanned to include the Anterior Extension Device and rigid link and patient's nose. A facial scan is taken while the patient is smiling. The rescanned bio-copy is aligned and merged with the facial scan on the basis of at least three points of reference on the patient's nose. A prosthetic device is designed using in combination the intraoral scans aligned with the facial scan.

### CROSS REFERENCE TO RELATED APPLICATIONS

This application claims priority to Provisional Patent Application No. 63/520,182 filed Aug. 17, 2023, the entire disclosure of which is hereby incorporated by reference and relied upon.

### BACKGROUND OF THE INVENTION

Field of the Invention. The invention relates generally to a method and apparatus for custom design of dental prosthetic appliances, and more particularly to the precise alignment of 3D intra-oral scans with a facial scan in the practice of dentistry and prosthodontics.

Description of Related Art. In the field of prosthodontics and cosmetic dentistry, addressing esthetic concerns is a primary focus. Conditions such as edentulism (tooth loss), fractured anterior teeth, and an unappealing smile pose significant psychological, functional, and esthetic challenges for patients. It is well-documented that effective prosthetic rehabilitation, coupled with enhanced cosmetic outcomes, significantly improves a patient's quality of life while reducing associated morbidity. The available dental prosthetics range widely, including options such as dental crowns, veneers, dentures, and implant-supported fixed prostheses. Achieving the highest quality in these prosthetics requires considerable effort and time in both design and production. Given the urgency that often accompanies cosmetic dental needs, it is crucial to minimize the time required to design, produce, and fit a prosthetic device-even a temporary one—to enhance patient satisfaction and outcomes.

Conventional methods for fabricating prosthetic devices have historically posed significant challenges, resulting in prolonged chair time, extended periods during which the patient experiences cosmetic concerns, and an increased risk of health complications. These issues underscore the need for more efficient and effective approaches in prosthetic fabrication.

The rise of digital dentistry has marked a pivotal paradigm shift. Several technologies have been introduced to address these challenges within the world of digital dentistry. Cutting-edge techniques like intra-oral scanning and facial scanning are examples of such introductions. Digital tools empower dental practitioners to capture accurate, meticulous impressions while circumventing the need for conventional and often cumbersome impression materials. Digital restoration design empowers providers to exert direct control over the quality of their restorations.

Many patients are overly eager to believe in the power of new technology. Digital tools imply, if not outright promise, swift fabrication and efficient and discomfort-free treatment options. That is to say, patient expectations in the efficacy of these new digital tools can be unrealistic. Despite the remarkable improvements offered by digital dentistry, there remain challenges posed by the integration of these techniques as well when it comes to patient communication and meeting patient expectations.

While the advancements in digital tools hold immense promise, there remain certain complexities and challenges that have muted their effectiveness. In particular, there are inherent inaccuracies involved when attempting to merge intraoral and facial scans.

Intraoral scans are typically produced using small, hand-held scanning devices specifically configured to capture and output graphic representations of the topographical contours inside the patient's mouth. The images are recorded in digital files, typically in STL format, stored in a non-transitory computer readable medium coded with instructions and executed by a processor to perform the scanning-related operations and display 3D image on a display screen.

Facial scanning, on the other hand, is an amazing digital tool that offers many promising advantages. 3D facial scanning provides detailed information about the patient's facial features, including the relationship between the teeth, jaws, lips, and surrounding soft tissues. Facial scans can be used to analyze facial proportions, symmetry, and other aesthetic factors to create personalized treatment plans. The ability to visualize the patient's face in 3D allows dentists to design dental restorations and orthodontic treatments that harmonize with the natural contours of the face, resulting in improved aesthetics and patient satisfaction.

By incorporating 3D facial scans into the treatment process, dentists can better predict how a proposed treatment will affect a patient's facial appearance. This knowledge leads to more accurate treatment planning. 3D facial scans offer a visual representation of the proposed treatment, making it easier for dentists to explain procedures to patients. Patients can better understand the changes that will occur, which enhances communication and helps manage expectations. And, 3D facial scan data can be shared easily with other dental specialists, such as orthodontists, prosthodontists, and maxillofacial surgeons, facilitating interdisciplinary collaboration for complex treatment cases.

However, all facial scanners today lack the capability of scanning teeth; facial scanners are unable to see behind lips with the level of detail required by the dental care professional. Therefore, a challenge in using facial scanning in dentistry is the ability to accurately merge the intraoral scan data with the facial scan data. This process, known as registration or alignment or stitching, involves aligning the intraoral and facial scans (and sometimes also a CBCT scan) to create a seamless and accurate 3D representation of the patient's entire dental and facial structures. However, achieving perfect alignment between intraoral and facial scans can be challenging due to various factors. One of the key issues is the distance of the facial scanner from the patient's face, which can lead to difficulties in accurately capturing detailed scans of the teeth. These scans are crucial as reference points for merging the data, and any inaccuracies can hinder the alignment process.

Misalignment can lead to inaccuracies in treatment planning and the creation of dental restorations. In particular, aligning the facial scan with the patient's teeth scan-particularly how their teeth occlude—is essential for designing effective cosmetic dental restorations and orthodontic treatments. This precise alignment ensures that the restorations and treatments are both functionally accurate and aesthetically pleasing, leading to better patient outcomes. Achieving accurate alignment of the occlusal surfaces from both facial and intraoral scans demands precision, attention to detail, and occasionally, a bit of luck. Successfully integrating the soft tissue information from the facial scan with the occlusal/bite registration from the intraoral scan is crucial for creating dental restorations that not only look natural but also accurately replicate the patient's natural smile.

Addressing these challenges requires ongoing advancements in scanning technology, improved software algorithms for automatic alignment, and continued education and training for dental professionals on the best practices for capturing and merging accurate facial and intraoral scans. Overcoming these challenges will further enhance the potential benefits of facial scanning in dentistry and its overall clinical utility.

Until more advanced technology becomes available, the current approach involves taking two facial scans: one with a retracted smile that reveals the teeth and one without. The prosthodontist or lab technician then aligns the facial scan with the retracted smile to the intraoral scan, followed by aligning the facial scan without the retracted smile to the one with the retracted smile. However, each alignment introduces the potential for errors, leading to misalignments between the intraoral scan and the facial scan. As a result, even if the dental surgeon creates what appears to be a beautiful outcome, the patient may be dissatisfied because the final result does not meet their expectations.

As the technology continues to advance and become more accessible, 3D facial scanning in dentistry holds great promise for enhancing treatment outcomes and patient experiences. Addressing the challenges is crucial to realize the full potential of this technology in dental practice.

There is therefore a need to overcome the current inaccuracies and inefficiencies, notably due to the limitations of facial scanners in capturing detailed information about teeth and the challenges of aligning facial scans with intraoral scans.

It is clear that the use of digital technology—namely computer-aided design enabled by 3D scanning—is the future of prosthetic dentistry. An obstacle to realizing the effective implementation of such digital tools lies in the accurate combination of the facial scan and intraoral scan, and in some cases also the CBCT scan.

### BRIEF SUMMARY OF THE INVENTION

The invention contemplates a method for custom designing and fabricating a dental prosthetic appliance for a patient having a face with a nose and a mouth. The mouth encloses an upper jaw and a lower jaw moveable into bite registry with one another. The upper jaw includes at least one central incisor or an edentulous site thereof. The method comprises the steps of creating an IOS-upper scan comprises a 3D intra-oral scan of the patient's upper jaw, and making a bio-copy of the IOS-upper scan. The bio-copy has an anterior section that includes at least one central incisor or the edentulous site thereof. The bio-copy is trimmed by deleting the anterior section. An Anterior Extension Device is affixed to the patient's upper jaw. The affixing step includes filling a tray portion of the Anterior Extension Device with curable bite registration material. A rigid link is located on the patient's face. The rigid link has a lower end and an upper end. The locating step includes positioning the upper end of the rigid link at the base of the patient's nose. A facial scan of the patient is produced using a facial scanner. The step of producing a facial scan includes capturing a 3D facial scan of the patient's nose. The bio-copy is re-scanned following the step of locating the rigid link on the patient's face. The re-scanning step includes filling the trimmed anterior section with a 3D scan capturing the Anterior Extension Device and the rigid link and the patient's nose. The re-scanned bio-copy comprises an IOS-nose scan. The IOS-nose scan and the IOS-upper scan form Multiple Aligned IOS files. Merging the facial scan and the Multiple Aligned IOS files to create a synchronized 3D file. The merging step includes aligning at least three common points of reference on the nose portion of the facial scan and the IOS-nose scan. A useful 3D file is created by removing from the synchronized 3D file at least the IOS-nose scan.

According to another aspect of the invention, an Anterior Extension Device is provided for temporary affixation to a patient's upper jaw. The Anterior Extension Device includes a tray portion. The tray portion has a bite plane adapted to cover the occlusal surface of at least one central incisor or an edentulous site thereof in the patient's upper jaw. The bite plane extends medially between lingual and labial ends. The distance between the lingual and labial ends represents the length of the bite plane. The bite plane extends laterally between left and right sides. The distance between the left and right sides represents the width of the bite plane. An inner wall extends upwardly from the lingual end of the bite plane. An outer wall extends upwardly from the labial end of the bite plane. A trans-labium cantilever portion extends from the outer wall to a free end adapted to project anteriorly outside the patient's mouth. A representation of a scannable 3D object is disposed on the free end. The width of the bite plane does not exceed 1.5 times the length so that the bite plane will cover the patient's incisor teeth and without covering any molar or pre-molar teeth.

An Anterior Extension Device for temporary affixation to a patient's upper jaw having incisor and molar and pre-molar teeth or edentulous sites thereof, said Anterior Extension Device comprising:

The method of this invention leverages the patient's nose as a unified reference point for merging and aligning intraoral and facial scans. The method transforms the way intraoral and facial scans are combined, significantly improving diagnostic precision, enhancing treatment planning, and facilitating superior patient communication.

The benefits of this method are extensive. It notably reduces the chance of errors by simplifying the scanning process, thus enhancing the accuracy and reliability of the combined model. This breakthrough is key to developing a detailed and precise representation of the patient's dental and facial anatomy, essential for devising effective treatments and achieving outstanding esthetics. Dental professionals are empowered to offer a clearer and more accurate visualization of the patient's future appearance, improving the quality of visual simulations presented during consultations.

The Anterior Extension Device facilitates the merging of the facial scan with the intraoral scan by incorporating the nose into the IOS-nose scan. This versatile device is universally applicable and can be adapted to fit any patient.

The method and device of this invention not only elevates the standard of dental treatments but also significantly boosts patient satisfaction by providing clear, attainable treatment goals. Furthermore, the method and device mark significant advancements in dental esthetics and prosthodontics. The provide a detailed perspective of the patient's dental and facial anatomy, enabling restorations that are both functionally sound and esthetically in tune with the patient's facial features. The invention facilitates the integration of commercially available merging software and 3D printing technology, thus advancing the shift toward a more patient-focused approach to prosthetic dentistry, heralding an era of personalized care designed to meet each patient's unique esthetic preferences.

### BRIEF DESCRIPTION OF THE SEVERAL VIEWS OF THE DRAWINGS

These and other features and advantages of the present invention will become more readily appreciated when considered in connection with the following detailed description and appended drawings, wherein:

FIG. 1 is a schematic diagram of the method of the invention according to an exemplary embodiment;

FIG. 2 depicts 3D intra-oral scans of the upper and lower jaws separately and also in bite registry;

FIG. 3 is a bio-copy of the intra-oral scan of the upper jaw, and having an Anterior section trimmed away;

FIG. 4 is a top view of an Anterior Extension Device according to an embodiment of the invention;

FIG. 5 is a side view of the Anterior Extension Device of FIG. 4;

FIG. 6 shows the trans-labium cantilever portion of the Anterior Extension Device extending outside the patient's mouth, and having first and second representations of incisor teeth;

FIG. 7 is a further progression of FIG. 6, showing a rigid link located between the Anterior Extension Device and the base of the patient's nose;

FIG. 8 represents a completed re-scan of the bio-copy, which includes the patient's nose;

FIGS. 9A and 9B illustrate the Multiple Aligned IOS files from differing perspectives;

FIG. 10 is a facial scan of the patient produced using a facial scanner;

FIG. 11 shows the merging step in which at least three points of reference on the nose portion of the facial scan are aligned with at least three points of reference on the nose portion of the IOS-nose scan;

FIG. 12 depicts the synchronized 3D file resulting from FIG. 11;

FIG. 13 shows the useful 3D file, which is the patient's facial scan in a natural smile is in perfect alignment with the Multiple Aligned IOS files; and

FIG. 14 illustrates a prosthetic dental device design that is in harmony with the patient's facial scan, ensuring both functional and aesthetic integration.

### DETAILED DESCRIPTION OF THE INVENTION

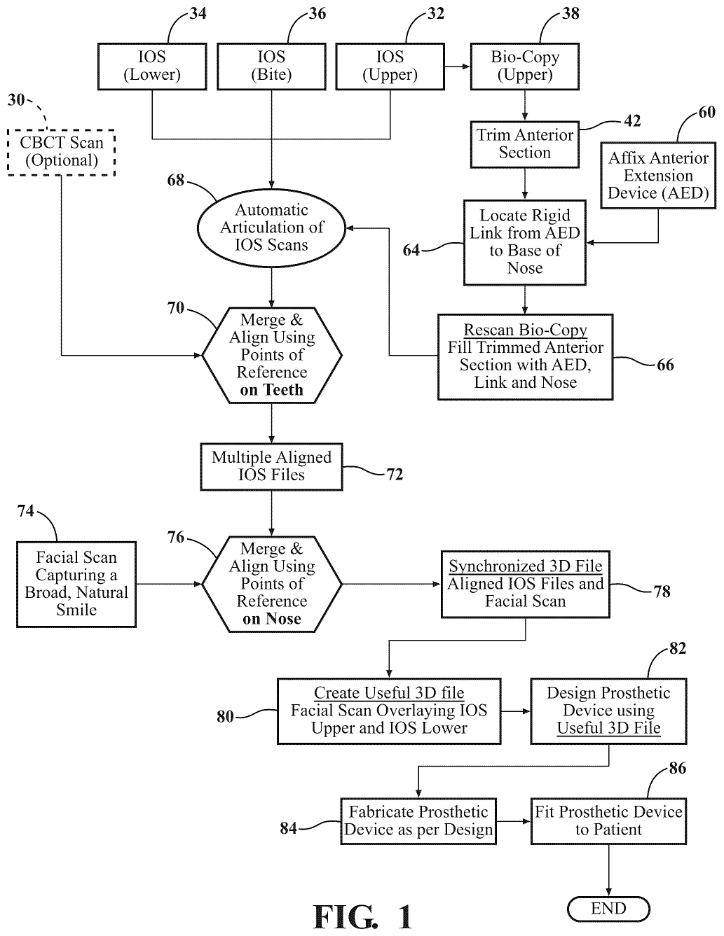

Referring to the figures, wherein like numerals indicate like or corresponding parts throughout the several views, a method and device for custom designing and fabricating a dental prosthetic appliance is shown and described. The method and device are intended primarily for human patients, although with minor adaptations within the skill of the ordinary veterinary surgeon, the method and device could be applied to animal dentistry. A detailed diagram of the method, according to one exemplary embodiment, appears in FIG. 1. Each step of the method will be described in due course.

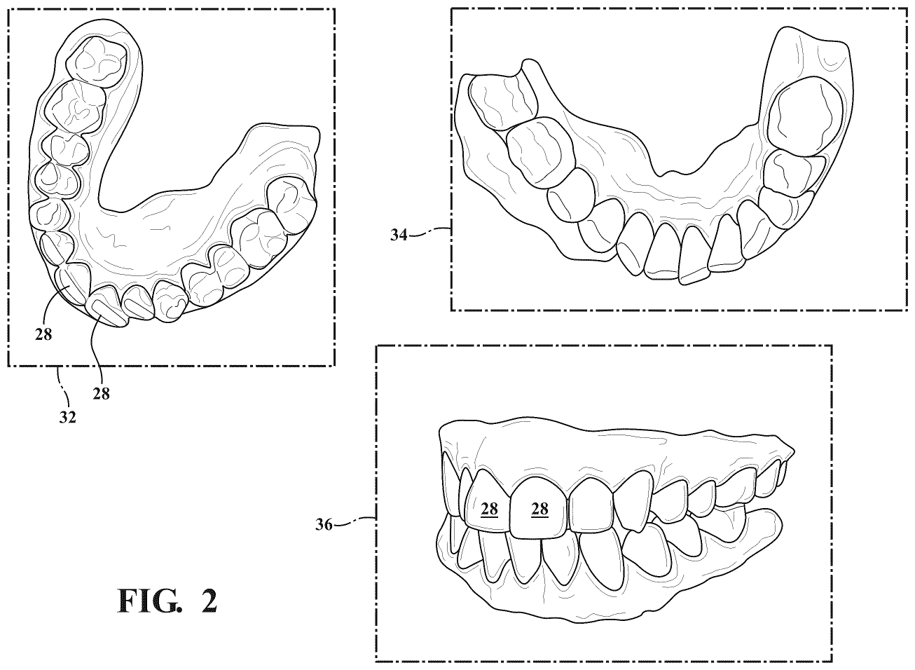

Some of the figures include depictions of a human patient having a typical face with a nose 20 and a mouth 22. The mouth 22 encloses an upper jaw and a lower jaw moveable into bite registry with one another in the normal fashion. In FIG. 2, 3D intra-oral scans of each upper and lower jaw are shown, together with a 3D intra-oral scan showing the jaws in bite registry. As is well known, teeth set in the upper jaw typically include two central incisors 28. In some cases, the patient could be missing one or both incisors 28, in which case one or two edentulous sites will appear where the central incisor(s) had previously resided.

Referring now to FIG. 1, the method is described according to a series of steps or actions. In some cases, it is not necessary that the steps be performed in the sequence suggested in FIG. 1. Thus, use of words like “next” and “then” are not intended to be sequentially limiting unless expressly stated otherwise. Moreover, it is not necessary that all of the steps be performed by the same person or machine, or in the same location, or within any set period of time. That is to say, the method shown in FIG. 1 could be performed in a modified sequence, by multiple people and/or machines working in various locations over a period of several weeks or even months.

The first step, indicated at 30, is an optional step that involves creating a CBCT scan. A CBCT scan 30 is a 3D cone-beam computed tomography image that captures at least a portion of the patient's facial bones. Typically, the CBCT scan 30 will capture both the upper and lower jawbones in their entirety, providing a comprehensive view of the patient's oral anatomy. In some cases, the attending physician may conclude that a CBCT scan 30 is not needed for the situation, in which case this step is omitted.

Steps 32, 34 and 36 correspond with FIG. 2. Step 32 is creating an IOS-upper scan. The IOS-upper scan 32 comprises a 3D intra-oral scan of the patient's upper jaw 24. Step 34 is creating an IOS-lower scan. The IOS-lower scan 34 comprises a 3D intra-oral scan of the patient's lower jaw 26. And step 36 is creating an IOS-bite scan. The IOS-bite scan 36 comprises a 3D intra-oral scan of the patient's upper and lower jaws in bite registry. The several intra-oral scans 32, 34, 36 can be performed by any one of several commercially available hand-held intra-oral scanning devices (not shown). Such intra-oral scanners are well-known to those of skill in this art and can be any suitable product of the type commercially available and sufficiently suited to the task. The intraoral scanner may have the capability to provide multiple scans for the same arch as a bio-copy or additional scan.

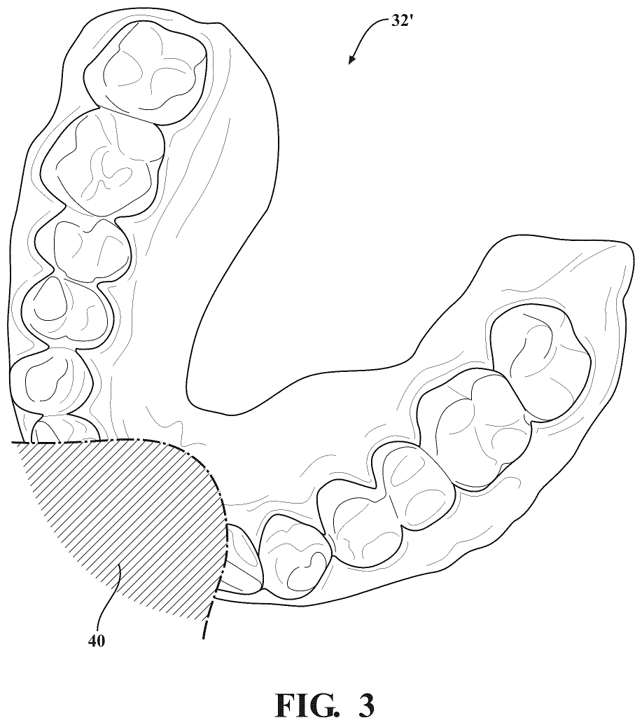

Step 38 is making a bio-copy 32′ of the IOS-upper scan 32. Step 38 corresponds with FIG. 3. Bio-copies are commonly used in dentistry for a variety of purposes, such as to copy a preexisting clinical situation, to create a translucent overlay during the design phase to help with the contour of the buccal corridor and occlusal morphology, and to create an exact digital scan of a tooth or teeth that can then itself be bio-copied, to name but a few. The bio-copy 32′ has an anterior section 40. The anterior section 40 refers to the specific region of the bio-scan 32′ that includes one or both central incisors 28, or the edentulous site(s) thereof. References to central incisors 28 are intended to include both natural and temporary or permanent dental prosthetics in that area.

The anterior region 40 may also include some or all of the lateral incisors. In some cases the anterior region 40 could even capture a portion of the canines. It being understood that the exact boundaries of the anterior region 40 are of lesser significance, but in most cases will not extend as far as the molars. Step 42 is trimming the bio-copy 32′ by deleting the anterior section 40, as suggested in FIG. 3. In other words, the anterior section 40 is culled from the bio-copy 32′, thus leaving a noticeable vacancy in its scan data.

After the bio-copy 32′ has been trimmed (step 42), in real life the surgeon temporarily affixes an Anterior Extension Device 44 to the patient's upper jaw 24, which is indicated at step 60 in FIG. 1. There are likely numerous suitable ways to temporarily seat, or affix, the Anterior Extension Device 44 to the patient's upper jaw 24. One convenient method is to use a quantity of curable bite registration material 61. Dentistry professionals use bite registration material 61 to make impressions, to ensure accurate alignment and fit of dentures, crowns and bridges, and for a host of other purposes. Various materials can be used for bite registration, including but not limited to: impression plasters, model compounds, vinyl polysiloxane impression material, and the like. In the illustrated examples, the Anterior Extension Device 44 is fastened to the patient's upper jaw using a suitable bite registration material 61, or some other fixation material suited to the task.

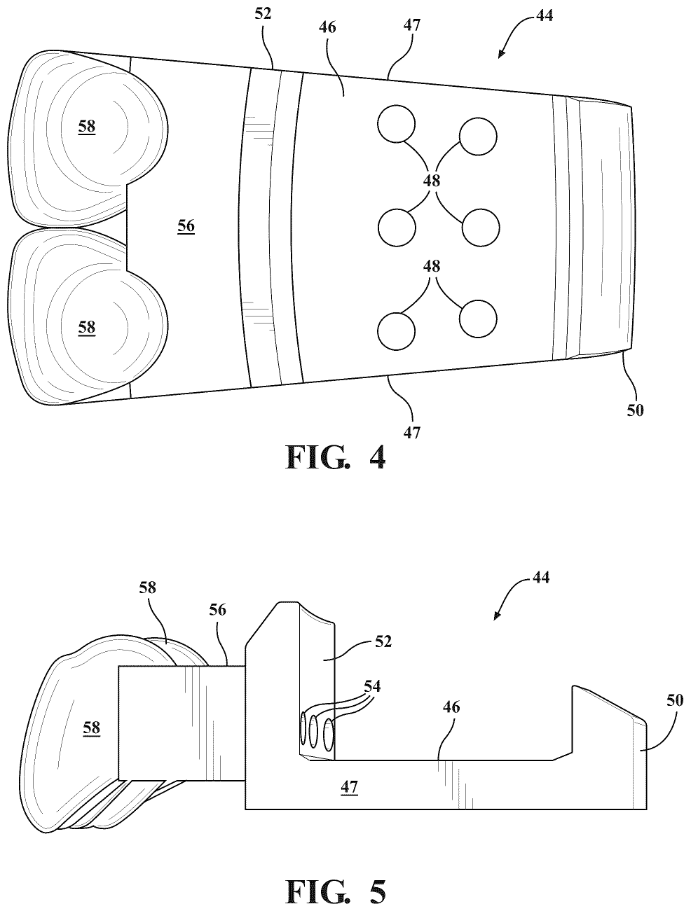

An exemplary embodiment of the Anterior Extension Device 44 is shown in FIGS. 4 and 5. The Anterior Extension Device 44 has a tray portion that is adapted to be removably affixed to one or both of the patient's upper central incisors 28, the edentulous site(s), or a temporary or permanent dental prosthetic in that area. The tray portion is a generally U-shaped member having a bite plane 46 adapted to cover the occlusal surface of the central incisors 28, or the edentulous sites thereof, in the patient's upper jaw 24. The bite plane 46 extends medially (i.e., parallel to the sagittal plane) between lingual and labial ends, and laterally (i.e., parallel to the coronal plane) between left and right sides 47. The distance between the lingual and labial ends represents a length of the bite plane 46, whereas the distance between the left and right sides 47 represents the width of the bite plane 46. In the top view of FIG. 4, it can be seen that the bite plane 46 could have a somewhat isosceles trapezoidal shape, in that the lingual and labial ends are generally parallel to one another, but the connecting sides 47 are skewed so that the labial end is slightly wider than the lingual end. In the preferred embodiment, width of the bite plane 46 at any point is limited to no more than 1.5 times the length. Generally speaking, narrower is better. The embodiment depicted in FIG. 4 shows the width of the bite plane 46 at its labial end (its maximum point) is about equal to its length.

The width of the left and right sides 47 is constrained in this way so as not to extend much, if at all, past the four upper incisor teeth. That is to say, by limiting the width of the bite plane 46 to less than or equal to 1.5 times the length, the left and right sides 47 are limited to covering some or all of the central 28 and lateral incisors, but preferably will not cover any significant portion of the canine/cusped teeth. It is preferred that the canine/cusped teeth not be covered by the bite plane 46. And in any event, the first and second premolars, as well as all of the molar teeth, remain fully exposed and uncovered by the bite plane 46. The motivation to restrict the lateral width of the bite plane 46 within the critical range of no more than 1.5 times the length is so that the Anterior Extension Device 44 will only cover the incisor teeth, and in extreme cases only a portion of canine/cusped teeth. The reason behind this motivation will become apparent in the description below.

At least one aperture 48, but preferably several apertures 48, are formed in the bite plane 46. The term aperture is used in the broadest possible sense to include through-holes as well as dimples (both concave and convex), ribs, scoring, and a variety of other types of surface treatments and formations effective to enhance mechanical grip between a surface and an adhesive. The at least one aperture 48 is configured to protrude into or receive therein a quantity of the uncured bite registration material 61. The apertures 48 thus facilitate adhesion of the bite registration material 61 to the Anterior Extension Device 44 when in its cured state.

An inner wall 50 extends upwardly from the lingual end of the bite plane 46. The inner wall 50 terminates at a given lingual height, the upper edge of which could be fashioned with a slant, or taper, away from the bite plane 46 as apparent from the side view of FIG. 5. The tapered upper edge increases comfort by softening the contact points with the patient's hard palate (roof of mouth 22). An outer wall 52 extends upwardly from the labial end of the bite plane 46. The outer wall 52 may be designed with a gentle labial curvature matching the curvature of the patient's anterior teeth in the upper jaw 24. At least one aperture 54, but preferably several apertures 54, are formed in the outer wall 52. Once again, the term aperture is used broadly to includes through-holes as well as dimples and ribs and various formations suitable to facilitate mechanical adhesive grip of the bite registration material 61 to the Anterior Extension Device 44. The upper edge of the outer wall 52 has a labial height and may be a tapered as seen in FIG. 5 to increase patient comfort. In the exemplary embodiment, the labial height is greater than the lingual height.

A trans-labium cantilever portion 56 extends from the outer wall 52. A free or distal-most end of the trans-labium cantilever portion 56 is designed to project anteriorly outside the patient's mouth 22. That is to say, the cantilever portion 56 passes through the patient's lips and terminates outside the mouth 22 at a free end on which is stationed a representation of a scannable 3D object. The representation of a scannable 3D object on the free end could be configured with any one of a variety of different shapes that could be captured by the intraoral scanner. In the illustrated examples, the free end of the cantilever portion 56 is shown having formations in the nature of first and second representations 58 of incisal teeth. The motivation to mimic incisal teeth 28 arises from the nature of the software commonly used to drive intraoral scanning devices. Such software is programmed to readily recognize tooth shapes. By configuring the free end with representations 58 that mimic incisal teeth 28, the software can easily register the shapes in 3-dimensional space. Although the illustrated examples depict first and second representations 58, i.e., two teeth, it will be understood that in some cases it may be sufficient to use only one representation 58 or some altogether different 3D shape that is scannable.

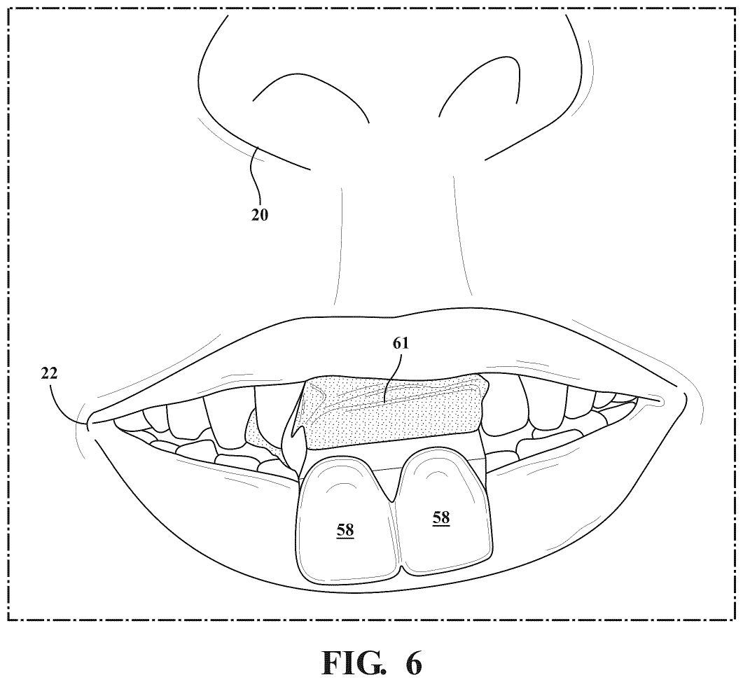

As mentioned above, one convenient method to temporarily affix the Anterior Extension Device 44 to the patient's upper jaw is to use a curable bite registration material 61. In such cases, the affixing step 60 includes filling the tray portion of the Anterior Extension Device 44 with curable bite registration material 61. The Anterior Extension Device 44 is then placed in the patient's mouth 22, such that the bite plane 46 covers the patient's incisors 28, or gums along the edentulous sites thereof. The trans-labium cantilever portion 56 passes through the gums, holding the first and second representations 58 outside the mouth 22, as shown in FIG. 6. While in its fluid state, the bite registration material 61 enters (or otherwise forms around) the apertures 48, 54, while at the same time flowing around the patient's incisors 28, the edentulous site(s), or a temporary or permanent dental prosthetic in that area. The patient may be instructed to maintain a moderate level of bite pressure against the bite plane 46 so that the Anterior Extension Device 44 remains pressed firmly in position. Once cured, the bite registration material 61 holds the Anterior Extension Device 44 in its set position.

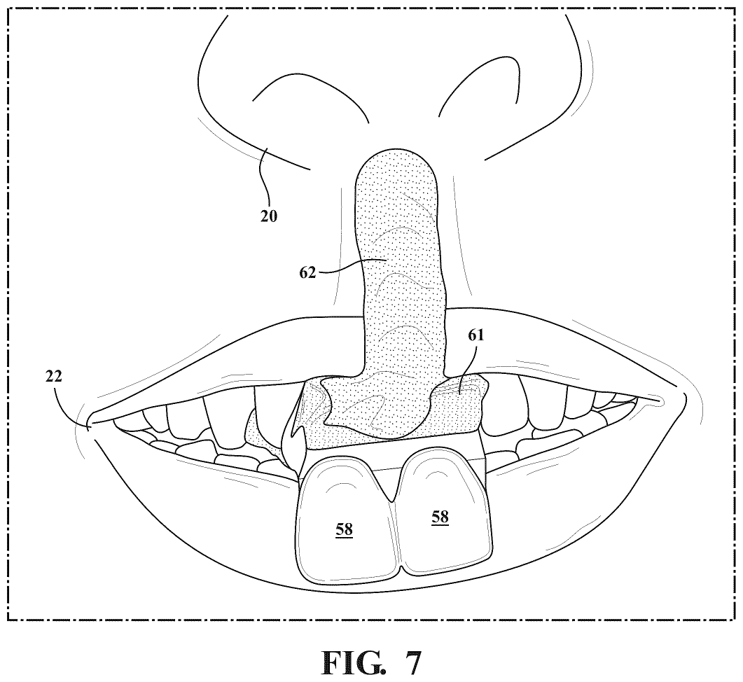

With the Anterior Extension Device 44 temporarily affixed, the method is continued with the step of locating a rigid link 62 on the patient's face (step 64). FIG. 7 corresponds with step 64. The link 62 can be made from any sufficiently rigid object or material. Given the ubiquitous availability of bite registration material 61 in dental offices, the locating step 64 may include extruding a bead of curable bite registration material 61 in order to form the rigid link 62 on-demand. Alternatively, the rigid link 62 could be made from paper board or plastic or any other suitable substance. While the exemplary embodiment envisions a disposable rigid link 62, it is recognized that the rigid link 62 could instead be made in a re-useable design.

Regardless of its substance or form, the rigid link 62 has a lower end and an upper end. The locating step 64 includes directly attaching the lower end to the trans-labium cantilever portion 56 of the Anterior Extension Device 44. The upper end of the rigid link 62 is located directly under, i.e., at the base of, the patient's nose 20, as shown in FIG. 7. In this manner the rigid link 62 serves as a stable static bridge between the Anterior Extension Device 44 and the patient's nose 20.

After the rigid link 62 has been located on the patient's face, the method continues by re-scanning the bio-copy 32′, which is step 66 in FIG. 1. Recall from step 42 that the anterior portion 40 of the bio-copy 32′ was deleted in a trimming operation. Now with the Anterior Extension Device 44 temporarily secured in position, the re-scanning step 66 seeks to fill in the trimmed anterior section 40 with a 3D scan capturing the Anterior Extension Device 44, as well as of the rigid link 62 and of the patient's nose 20. The re-scanning step 66 is initiated inside the patient's mouth 22, where existing scanned areas (FIG. 3) are quickly recognized by the software, providing perfect alignment registry with the original bio-copy 32′. The scanning software thus automatically—indeed seamlessly—fills the trimmed anterior section 40 with capture of the tray portion of the Anterior Extension Device 44.

As mentioned, the width of the left and right sides 47 is controlled so as not to extend much, if any, past the upper incisor teeth. The motivation to restrict the lateral width of the bite plane 46 to cover only the incisor teeth, and in extreme cases only a portion of canine/cusped teeth, is so that existing scanned areas are exposed for recognition by the software, thereby facilitating alignment registry with the original bio-copy 32′. In a perfect scenario, the width of the bite plane 46 corresponds closely to the width of the trimmed anterior section 40, with the molars, pre-molars and preferably also the canines remaining exposed for scan capture even though the Anterior Extension Device 44 is seated in the patient's mouth.

Intraoral scanners perform best capturing uninterrupted spans of static surfaces. Lips are most definitively not static surfaces, and thus have in the past posed in insurmountable barrier to extension of an intraoral scan outside the mouth 22. Like a great reef to an ocean vessel, the lips heretofore blocked all attempts to expand an intraoral scan to the face. The Anterior Extension Device 44 and the rigid link 62 provide uninterrupted spans of static surfaces that act as a safe, scannable pathway to allow an intraoral scanner to extend the intra-oral scan outside the patient's mouth 22.

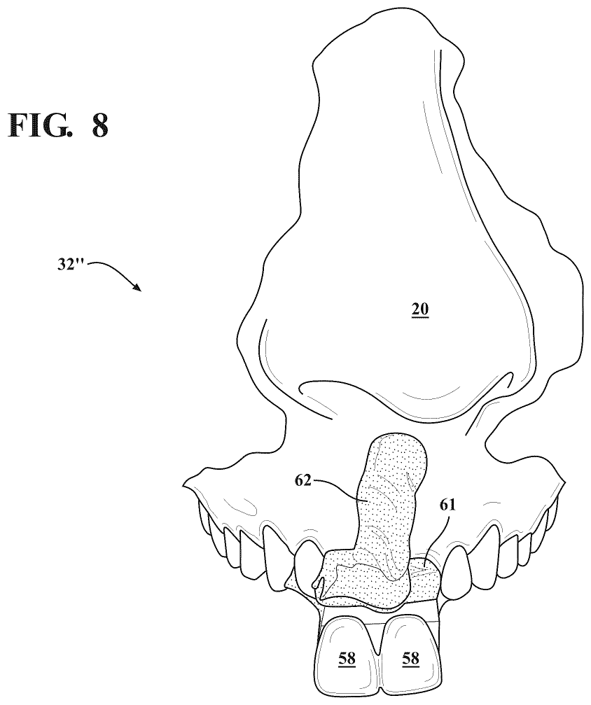

The re-scanning step 66 continues along the trans-labium cantilever portion 46, thus passing around the dynamic lips. The re-scanning step 66 progresses to include the first and second representations 58, and then onto the rigid link 62. The rigid link 62 is a key component of this invention, in that it provides a safe, scannable pathway to allow the intraoral scan to reach the patient's nose 20. Interestingly, the nose 20 is sufficiently static to be naturally scannable by an intraoral scanner. That is to say, without any prophylactic, an intra-oral scanner is able to capture a 3D representation of the patient's nose 20. Thus having reached the base of the nose 20 via the rigid link 62, the re-scanning step 66 continues with a capture of the patient's nose 20. The completed re-scan of the bio-copy, which now includes the patient's nose 20, is indicated at 32″ in FIG. 8. For convenience, the completed re-scan of the trimmed bio-copy 32′ will be referred to as the IOS-nose scan 32″.

Most commercially available, professional-grade, intra-oral scanning software will automatically articulate the IOS-nose scan 32″ and the IOS-upper scan 32 and the IOS-lower scan 34 based on the IOS-bite scan 36. In dentistry, the term articulated refers to the precise alignment and relationship between the upper and lower jaws. When scans or models are articulated, they accurately reflect how the jaws fit together, including the contact points of the teeth during biting or chewing. This alignment is crucial for creating functional and comfortable dental restorations. In the context of digital 3D scans, the intraoral scanner captures a detailed 3D image of both the upper and lower jaws (i.e., (IOS-upper 32 and IOS-lower 34), as well as their bite relationship (IOS-bite 36). The IOS-bite scan 36 accurately depicts the precise positioning and articulation of the jaws. The IOS-nose scan 32″, which includes the nose 20 and serves as an exact replica of the upper jaw 24, is automatically aligned or articulated correctly with the lower jaw as determined by the IOS-bite scan 36. The automatic articulation of these several intraoral scans or models is indicated at 68 in FIG. 1.

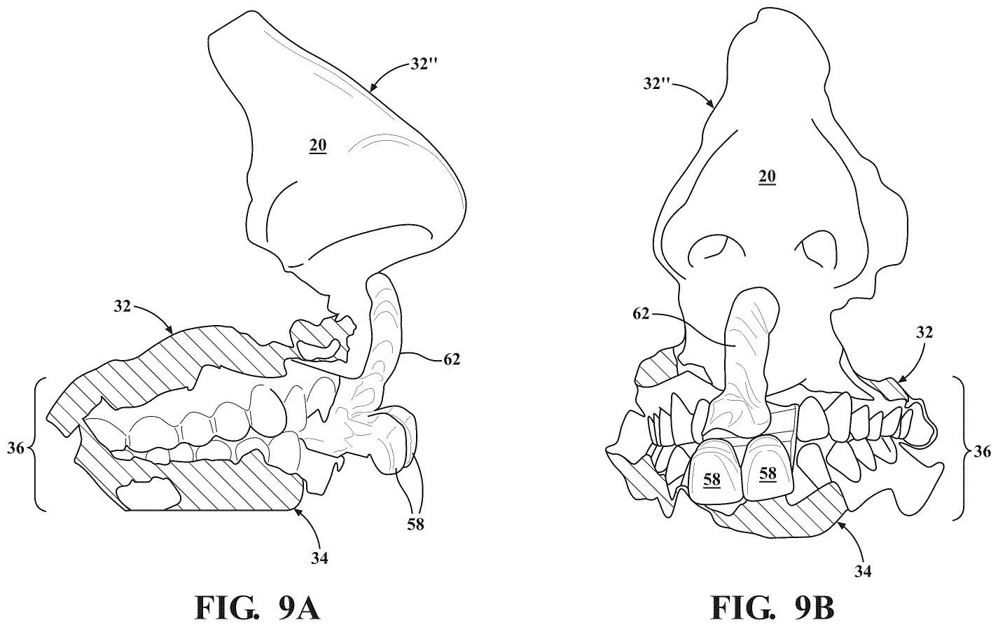

If a CBCT scan 30 has been created, it can now be merged with the articulated IOS scans at step 70. Substantially perfect alignment of the CBCT scan 30 with the articulated IOS scans is accomplished using points of reference on the teeth. That is to say, at multiple discrete locations, the operator or the software identifies a specific point on a tooth appearing in the CBCT scan 30 and the same specific point on the same tooth in the articulated IOS scans. The software is then able to perfectly orient the CBCT scan 30 relative to the articulated IOS scans, so that all of these scans 30, 32, 32″ and 34 are in matched alignment. The result is the formation of Multiple Aligned IOS files, indicated at step 72 in FIG. 1. In cases where there is not a CBCT scan 30, the articulated IOS scans will themselves comprise the Multiple Aligned IOS files. FIGS. 9A and 9B illustrate the resulting Multiple Aligned IOS files from different perspectives.

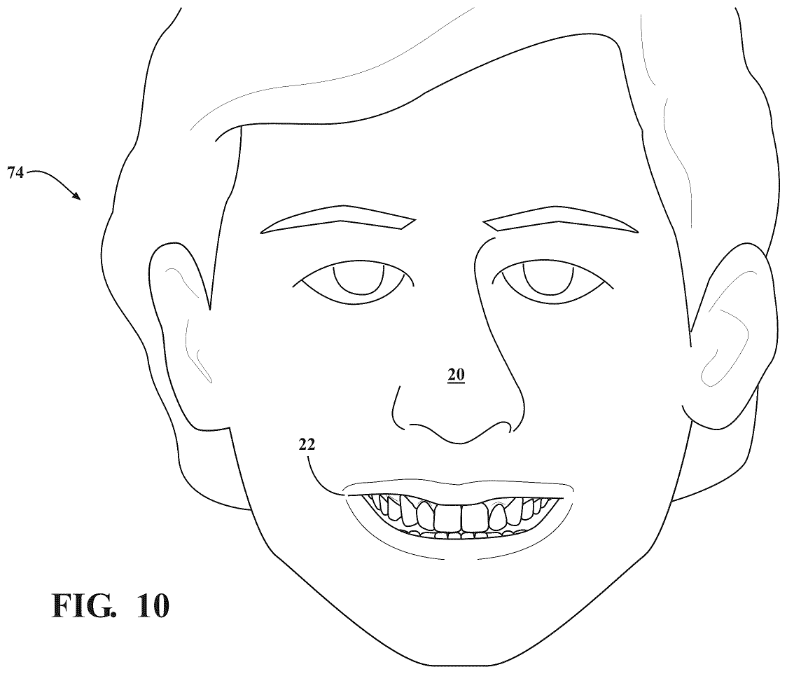

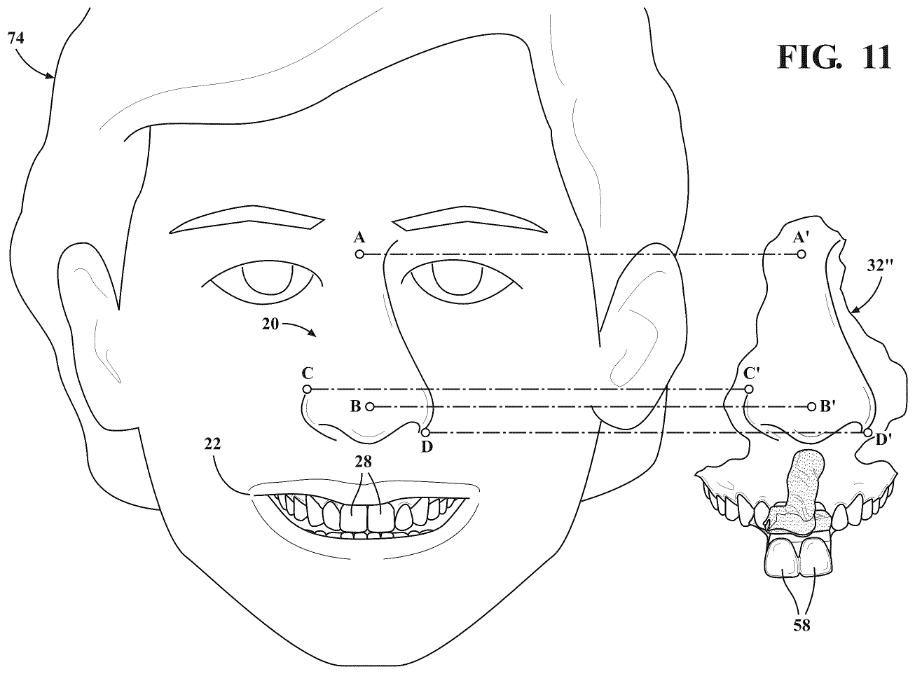

In FIG. 1, step 74 is producing a facial scan of the patient using a facial scanner. Step 74 corresponds with FIG. 10. A facial scanner is an altogether different kind of tool compared with an intraoral scanner. Facial scanners cannot capture the teeth precisely. The step of producing a facial scan 74 includes digitally capturing (extra-orally) the patient's mouth 22 formed in a broad natural smile, including fully the patient's nose 20. An exemplary facial scanner is the VECTRA H2 facial scanner.

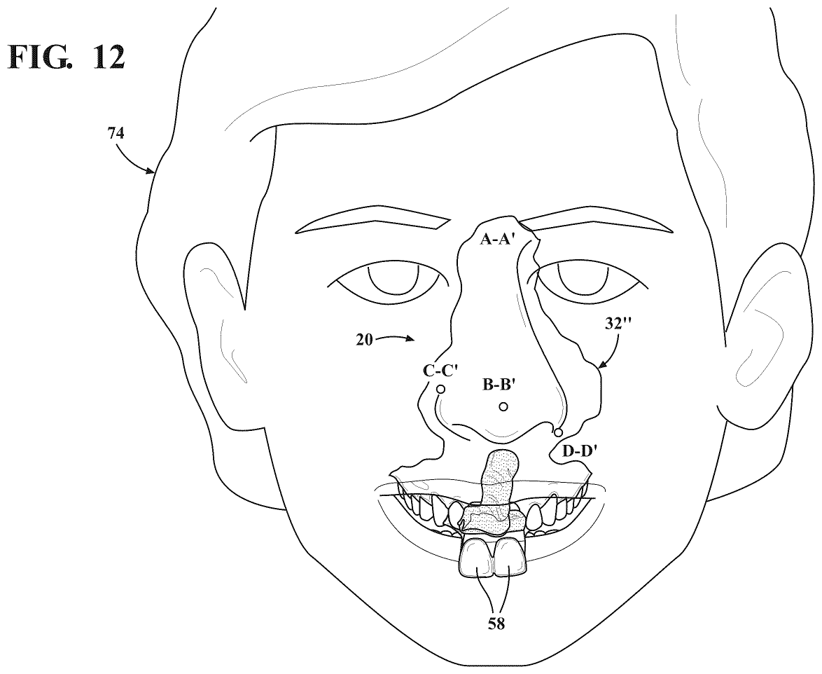

At step 76, the facial scan 74 is merged and aligned with the Multiple Aligned IOS files 72 to create a synchronized 3D file 78. Step 76 corresponds with FIGS. 11 and 12. The merging step 76 includes aligning at least three points of reference on the nose portion of the facial scan 74 with at least three points of reference on the nose portion of the IOS-nose scan 32″. In FIG. 11, the facial scan 74 appears on the left and the IOS-nose scan 32″ on the right. Using a Graphic User Interface (not shown), the operator identifies at least three points of reference on the nose portion of the facial scan 74. In this example, four points of reference are selected for improved accuracy: A, B, C & D. Point A is high on the nasal bridge; Point B is low on the nasal bridge near the tip; Point C is patient's right ala; and Point D is on the patient's left ala. Then turning to the IOS-nose scan 32″, the same four points of reference are digitally selected: A′, B′, C′ and D′. Selecting even more points (e.g., 5 or more) will further improve alignment accuracy.

With the points of reference (A-A′, B-B′, C-C′ and D-D′) duly connected, the software is able to perfectly orient the facial scan 74 relative to the IOS-nose scan 32″, and thus by extension to the entire set of Multiple Aligned IOS files 72, so that all of these scans 72 and 74 are in matched alignment. One example of software capable of performing this operation is marketed under the trademark Exocad®, available from exocad GmbH (Darmstadt, Germany). The result is the synchronized 3D file 78, shown in FIG. 12.

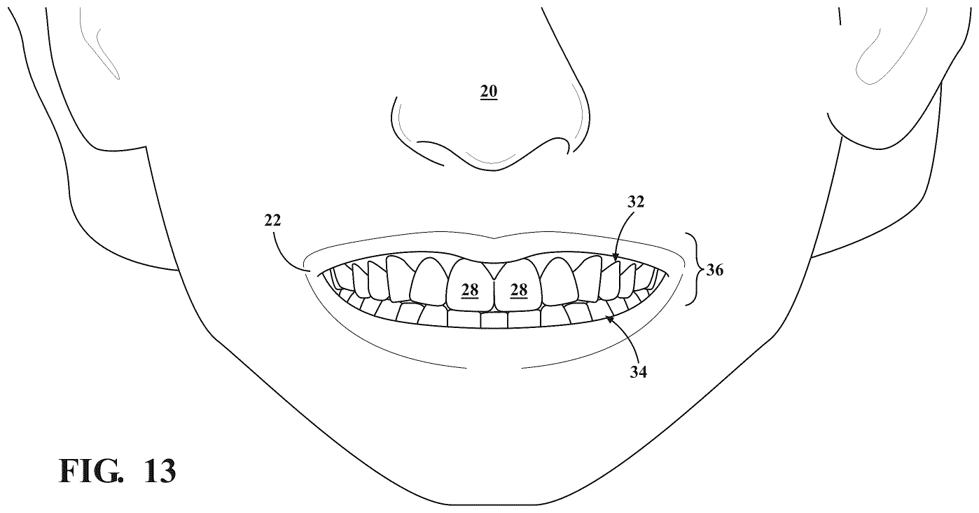

In step 80, a useful 3D file is created by removing from the synchronized 3D file 78 at least the IOS-nose scan 32″, which is the re-scanned bio-copy. FIG. 13 depicts the useful 3D file 80, in which the patient's facial scan 74 in a natural smile is perfectly aligned with the Multiple Aligned IOS files 72.

With this extraordinarily valuable piece of information, the practitioner can proceed to the step of designing a prosthetic dental device using the useful 3D file, indicated as step 82 in FIG. 1. Again, the Exocad® software can be used for this step. The completed design can be merged with the facial scan 74 to show the patient and communicate with them the treatment plan. Together, the dentist and patient can evaluate the amount of teeth showing, placement of the midline, lip support, etc., and eventually approve the treatment plan.

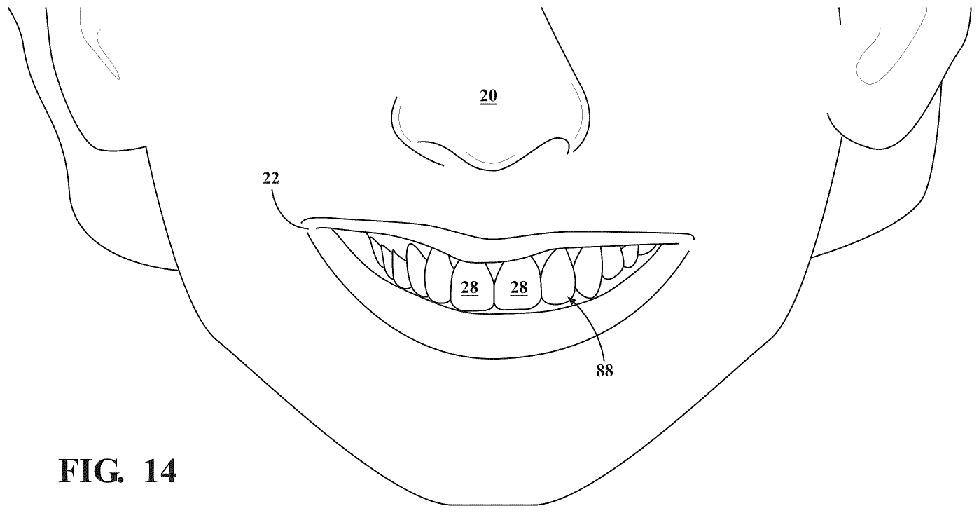

The designing step 82 concludes with generating a digital design file that can be sent to a 3D printer used to quickly fabricate a temporary prosthetic device. Step 84. Within minutes or hours, the patient can be fit with a temporary prosthetic dental device based on the digital design file. Step 86. Meanwhile, the digital design file can also be sent to a professional production facility capable of producing the highest quality permanent prosthetic dental device, which of course is eventually fit to the patient. FIG. 14 depicts a prosthetic dental device 88 (temporary or permanent) fabricated using a digital file created from the design step 82, and fit to the patient.

To summarize the overall steps of the method, a series of digital intraoral scans are taken of the patient, capturing the upper and lower arches as well as a bite registration. The Anterior Extension Device 44 is then utilized. Impression material is placed in the tray portion, which is inserted into the patient's mouth 22. A rigid link 62 is located on the patient's face, such as by extrusion of bite registration material 61, extending between the Anterior Extension Device 44 and base of the nose 20. Meanwhile, a bio-copy 32′ of the existing upper scan 32 is moved to new scan box. In this bio-copy 32′, the data overlaying where the Anterior Extension Device 44 will cover is isolated and removed, i.e., trimmed, allowing for a focused re-scan of only the missing information. Once the impression material 61 is set, the trimmed bio-copy 32′ is rescanned to finish the region now occupied by the attached Anterior Extension Device 44. But this re-scan continues to include the rigid link 62 and the patient's nose 20, thereby completing the data acquisition. By the end of this procedure, the dental team has obtained the following scans: the upper 32 and lower 34 arches, the bite registration 36, and the IOS-nose scan 32″ which contains the upper arch with the Anterior Extension Device 44 and the nose 20. All these scans are accurately aligned in occlusion as Multiple Aligned IOS files 72. At this stage, a facial scan 74 is taken while the patient is smiling. With the previously acquired scan of the patient's nose 20 from the IOS-nose scan 32″, the dental team can align and merge the nose portion of the facial scan 74 with the corresponding nose portion from the IOS-nose scan 32″. This seamless integration successfully aligns the facial scan with the intraoral scan, providing a comprehensive and precise representation of the patient's dental and facial structures for advanced treatment planning and aesthetic assessments.

The method of this invention can be characterized by leveraging the nose 20 as a unified reference point for both the intraoral 72 and facial 74 scans. The method of this invention significantly streamlines the scan file merging process into a singular, efficient operation that is more predictable than prior art methods. It is no longer important that the facial scanner cannot capture the teeth scan precisely. Using this invention, medical professionals can communicate better with their patients and lab technicians. They can present better treatment plans and provide better outcomes. The method can be practiced with commercially available facial scanners and intraoral scanners. In fact, this method will enable practitioners to utilize lower cost facial scanners while obtaining performance equivalent to high-end models. The method requires only one face scan and has the great advantage of reducing the amount of merging and errors.

The invention offers the ability for a more personalized care model that aligns closely with patients' esthetic expectations and builds greater confidence in the treatment process. Additionally, incorporating the nose 20 into every new patient's diagnostic comprehensive examination holds the potential for future benefits. Should a patient lose their teeth, the ability to merge scans based on the nose 20 ensures that dental professionals can still achieve precise alignments, underscoring the lasting value of this approach incomprehensive digital dentistry. By enabling more accurate alignments of these crucial diagnostic tools, the technique introduced marks a significant milestone in enhancing treatment planning, outcomes, and patient satisfaction in esthetic dentistry.

The foregoing invention has been described in accordance with the relevant legal standards, thus the description is exemplary rather than limiting in nature. Variations and modifications to the disclosed embodiment may become apparent to those skilled in the art and fall within the scope of the invention.

What is claimed is:

1. A method for custom designing and fabricating a dental prosthetic appliance for a patient having a face with a nose and a mouth, the mouth enclosing an upper jaw and a lower jaw moveable into bite registry with one another, the upper jaw including at least one central incisor or an edentulous site thereof, said method comprising the steps of:
creating an IOS-upper scan comprising a 3D intra-oral scan of the patient's upper jaw,
making a bio-copy of the IOS-upper scan, the bio-copy having an anterior section that includes at least one central incisor or the edentulous site thereof, trimming the bio-copy by deleting the anterior section,
affixing an Anterior Extension Device to the patient's upper jaw, said affixing step including filling a tray portion of the Anterior Extension Device with curable bite registration material,
locating a rigid link on the patient's face, the rigid link having a lower end and an upper end, said locating step including positioning the upper end of the rigid link at the base of the patient's nose,
producing a facial scan of the patient using a facial scanner, said step of producing a facial scan including capturing a 3D facial scan of the patient's nose,
re-scanning the bio-copy following said step of locating the rigid link on the patient's face, said re-scanning step including filling the trimmed anterior section with a 3D scan capturing the Anterior Extension Device and the rigid link and the patient's nose, the re-scanned bio-copy comprising an IOS-nose scan,
articulating the IOS-nose scan and the IOS-upper scan to form Multiple Aligned IOS files,
merging the facial scan and the Multiple Aligned IOS files to create a synchronized 3D file, said merging step including aligning at least three common points of reference on the nose portion of the facial scan and the IOS-nose scan,
creating a useful 3D file by removing from the synchronized 3D file at least the IOS-nose scan.

2. The method of claim 1, wherein the Anterior Extension Device has a trans-labium cantilever portion projecting anteriorly outside the mouth, said locating step including directly attaching the lower end of the rigid link to the trans-labium cantilever portion of the Anterior Extension Device.

3. The method of claim 2, wherein said locating step includes extruding a bead of curable bite registration material.

4. The method of claim 1, further including the step of creating a CBCT scan comprising a 3D cone-beam computed tomography image of at least a portion of the patient's mouth, and integrating the CBCT scan with the Multiple Aligned IOS files using multiple points of reference on the teeth.

5. The method of claim 1, wherein said step of producing a facial scan includes digitally capturing the mouth formed in a broad natural smile.

6. The method of claim 1, further including the step of creating an IOS-lower scan comprising a 3D intra-oral scan of the patient's lower jaw, and creating an IOS-bite scan comprising a 3D intra-oral scan of the patient's upper and lower jaws in bite registry, said articulating step including the IOS-lower scan and the IOS-bite scan to form the Multiple Aligned IOS files.

7. The method of claim 1, further including the step of designing a prosthetic dental device using the useful 3D file, said designing step including generating a digital design file.

8. The method of claim 7, further including the step of sending the digital design file to a 3D printer, fabricating a prosthetic dental device based on the digital design file.

9. The method of claim 8, further including the step of fitting the prosthetic dental device to the patient.

10. A method for custom designing and fabricating a dental prosthetic appliance for a patient having a face with a nose and a mouth, the mouth enclosing an upper jaw and a lower jaw moveable into bite registry with one another, the upper jaw including at least one central incisor or an edentulous site thereof, said method comprising the steps of:
creating an IOS-upper scan comprising a 3D intra-oral scan of the patient's upper jaw,
making a bio-copy of the IOS-upper scan, the bio-copy having an anterior section that includes at least one central incisor or the edentulous site thereof, trimming the bio-copy by deleting the anterior section,
affixing an Anterior Extension Device to the patient's upper jaw, said affixing step including filling a tray portion of the Anterior Extension Device with curable bite registration material, the Anterior Extension Device having a trans-labium cantilever portion projecting anteriorly outside the mouth,
extruding a bead of curable bite registration material to form a rigid link on the patient's face, the rigid link having a lower end and an upper end, said extruding step including directly attaching the lower end of the rigid link to the trans-labium cantilever portion of the Anterior Extension Device, said extruding step including positioning the upper end of the rigid link at the base of the patient's nose,
producing a facial scan of the patient using a facial scanner, step of producing a facial scan includes digitally capturing the mouth formed in a broad natural smile, said step of producing a facial scan including capturing a 3D facial scan of the patient's nose,
re-scanning the bio-copy following said step of locating the rigid link on the patient's face, said re-scanning step including filling the trimmed anterior section with a 3D scan capturing the Anterior Extension Device and the rigid link and the patient's nose, the re-scanned bio-copy comprising an IOS-nose scan,
articulating the IOS-nose scan and the IOS-upper scan to form Multiple Aligned IOS files,
merging the facial scan and the Multiple Aligned IOS files to create a synchronized 3D file, said merging step including aligning at least three common points of reference on the nose portion of the facial scan and the IOS-nose scan,
creating a useful 3D file by removing from the synchronized 3D file at least the IOS-nose scan,
designing a prosthetic dental device using the useful 3D file, said designing step including generating a digital design file,
fabricating a prosthetic dental device based on the digital design file, and
fitting the prosthetic dental device to the patient.

11. The method of claim 10, further including the step of creating an IOS-lower scan comprising a 3D intra-oral scan of the patient's lower jaw, and creating an IOS-bite scan comprising a 3D intra-oral scan of the patient's upper and lower jaws in bite registry, said articulating step including the IOS-lower scan and the IOS-bite scan to form the Multiple Aligned IOS files.

12. An Anterior Extension Device for temporary affixation to a patient's upper jaw having incisor and molar and pre-molar teeth or edentulous sites thereof, said Anterior Extension Device comprising:
a tray portion, said tray portion including a bite plane adapted to cover the occlusal surface of at least one central incisor or an edentulous site thereof in the patient's upper jaw, said bite plane extending medially between lingual and labial ends, the distance between said lingual and labial ends representing a length of said bite plane, the bite plane extending laterally between left and right sides, the distance between said left and right sides representing a width of said bite plane, an inner wall extending upwardly from said lingual end of said bite plane, an outer wall extending upwardly from said labial end of said bite plane,
a trans-labium cantilever portion extending from said outer wall to a free end adapted to project anteriorly outside the patient's mouth,
a representation of a scannable 3D object disposed on said free end, and
wherein said width of said bite plane does not exceed 1.5 times said length so that said bite plane will cover the patient's incisor teeth and without covering any molar or pre-molar teeth.

13. The Anterior Extension Device of claim 12, further including a rigid link having a lower end and an upper end, said lower end attached directly to said trans-labium cantilever portion, said upper end configured to terminate at the base of the patient's nose.

14. The Anterior Extension Device of claim 12, wherein said representation of a scannable 3D object on said free end comprises a first representation of an incisal tooth.

15. The Anterior Extension Device of claim 14, wherein said representation of a scannable 3D object disposed on said free end further includes a second representation of an incisal tooth disposed on said free end directly adjacent said first representation of an incisal tooth.

16. The Anterior Extension Device of claim 12, wherein said bite plane includes at least one aperture configured to receive a quantity of curable bite registration material.

17. The Anterior Extension Device of claim 12, wherein said outer wall includes at least one aperture configured to receive a quantity of curable bite registration material.

18. The Anterior Extension Device of claim 12, wherein said outer wall has a labial curvature.

19. The Anterior Extension Device of claim 12, wherein said inner wall has an upper edge and said outer wall has an upper edge, at least one of said upper edges being tapered.

20. The Anterior Extension Device of claim 12, wherein said inner wall has an upper edge terminating at a lingual height, and said outer wall has an upper edge terminating at a labial height, said labial height being greater than said lingual height.
